(19) United States
(12) Patent Application Publication
(10) Pub. No.: US 2003/0220486 A1
(43) Pub. Date: Nov. 27, 2003

(54) Mixed backbone oligonucleotides containing pops blocks to obtain reduced phosphorothioate content

(76) Inventors: Wen-Qiang Zhou, Laval (CA); Sudhir Agrawal, Shrewsbury, MA (US)

Correspondence Address: HALE AND DORR, LLP, 60 STATE STREET, BOSTON, MA, 02109

(21) Appl. No.: 10/291,058

(22) Filed: Nov. 8, 2002

Related U.S. Application Data
(63) Continuation of application No. 09/283,431, filed on Apr. 1, 1999.

(51) Int. Cl. C07H 21/04

(52) U.S. Cl. 536/23.2

(57) ABSTRACT

Mixed-backbone oligonucleotides POPS blocks have been designed and studied for their target affinity, nuclease stability in vitro and in vivo, Rnase H-activation properties, and their effect on phosphorothioate-related prolongation of partial thromboplastin time, in an effort to have agents with improved antisense activity with reduced phosphorothioate content.

This is a continuation-in-part of U.S. provisional application serial No. 60/080321, filed Apr. 1, 1998.

### BACKGROUND OF THE INVENTION

1. Field of the Invention

The invention relates to antisense oligonucleotides. In particular, the invention relates to modified antisense oligonucleotides having reduced sulfur content.

2. Summary of the Related Art

Mixed-backbone oligonucleotides (MBOs) provide a handle on modulating the pharmacological, pharmacodynamic, and pharmacokinetic profiles of antisense oligonucleotides. MBOs are currently the best choice as second-generation oligonucleotides over PS-oligos. MBOs contain appropriately placed segments of phosphorothioate oligodeoxynucleotide (PS-oligo) and one or more other type of modified oligodeoxynucleotide or oligoribonucleotide. The advantage of MBOs is that, while they retain the advantages of PS-oligo's stability against nuclease and Rnase H activation, the side effects inherent in PS-oligos (immune stimulation, complement activation and prolongation of partial thromboplastin time, etc.) can be minimized, depending on the nature of modified segment incorporated in MBOs. The positioning of the segments of modified oligodeoxynucleotides or oligoribonucleotides in a MBO may strongly affect its desired properties. In end-modified MBOs, a segment of PS-oligo is placed in the center to provide the RNase H activation, and segments of other type of modified oligonucleotide are placed at one or both of the 3′- and 5′-ends to modulate other antisense properties. End-modified MBOs have proved to be more effective than the PS-oligos as antisense agents and are currently being evaluated in clinical trials as therapeutic agents.

In certain end-modified MBOs, the existence and nature of modifications at the 2′-position of some nucleosides is important in providing increased duplex affinity and stability towards nucleases. The 2′-O-methylribonucleoside phosphorothioate and the 2′-O-methoxyethoxyribonucleoside phosphodiester are two types of modified nucleotide segments that have been studied most extensively. Incorporation of 2′-O-methylribonucleoside in the MBOs can increase the duplex stability with the target RNA. However, for an increase in nuclease stability, phosphorothioate internucleotide linkages are usually required as 2′-O-methylribonucleoside phosphodiester segments showed reduced nuclease stability. Incorporation of 2′-O-methoxyethoxyribonucleoside also provides an increase in duplex stability, and also demonstrated, in vitro, increased nuclease stability even with phosphodiester internucleotide linkages. Both of these types of end-modified MBOs have reduced the PS-oligo-related side effects. Differences in their pharmacokinetic and elimination profiles have been observed, however. The MBOs containing 2′-O-methylribonucleoside phosphorothioate show tissue distribution profiles similar to those of PS-oligos following intravenous administration with a significant improvement in stability and retention in tissues; the MBOs containing 2′-O-methoxyethoxyribonucleoside phosphodiester showed rapid elimination in urine and disposition in kidneys compared to PS-oligo.

There is a need for additional types of MBOs, which can significantly reduce the PS content without compromising the antisense properties, such as duplex stability, nuclease stability, Rnase H activity, antisense-based biological activity and tissue disposition. Ideally, such MBOs could be obtained by subtle modifications of the best MBOs available to date.

### BRIEF SUMMARY OF THE INVENTION

The invention relates to antisense oligonucleotides. In particular, the invention relates to modified antisense oligonucleotides having reduced sulfur content. The invention provides new MBOs, which have significantly reduced PS content without compromising their antisense properties, such as duplex stability, nuclease stability, Rnase H activity, antisense-based biological activity and tissue disposition. These new MBOs are obtained by subtle modifications of the best MBOs available to date.

In a first aspect, the invention provides oligonucleotides containing POPS blocks. POPS blocks are oligonucleotide regions containing alternating nucleoside phosphodiesters (PO) and nucleoside phosphorothioates (PS). In certain preferred embodiments, such nucleoside phosphodiesters and nucleoside phosphorothioates alternate in a one-to-one manner, i.e., PO-PS-PO-PS-PO-PS. In other preferred embodiments, such nucleoside phosphodiesters and nucleoside phosphorothioates alternate in a two-to-one PO to PS manner (PO-PO-PS-PO-PO-PS) or in a two-to-one PS to PO manner (PS-PS-PO-PS-PS-PO). In still other preferred embodiments, such nucleoside phosphodiesters and nucleoside phosphorothioates alternate in a two-to-two manner (PS-PS-PO-PO) or in a three-to-three manner (PS-PS-PS-PO-PO-PO). In yet additional preferred embodiments, the alternation of such nucleoside phosphodiesters and nucleoside phosphorothioates is irregular, provided however, that in such embodiments, a ratio of nucleoside phosphodiesters and nucleoside phosphorothioates of from 1:3 to 3:1 is maintained in at least one POPS block.

In a second aspect, the invention provides hybrid oligonucleotides comprising one or more POPS block. Hybrid oligonucleotides are described in U.S. Pat. No. 5,652,355, which is hereby incorporated by reference. Generally, such hybrid oligonucleotides comprise at least one region of deoxyribonucleoside phosphodiesters or phosphorothioates, which is flanked by regions of 2′-O-substituted nucleosides, which may be connected to each other and to the region of deoxyribonucleoside phosphodiesters or phosphorothioates by any type of internucleoside linkage. Thus, in this aspect of the invention, the invention comprises the improvement in a hybrid oligonucleotide of having one or more POPS block as a region of deoxyribonucleoside phosphodiesters or phosphorothioates.

In a third aspect, the invention provides inverted hybrid oligonucleotides comprising one or more POPS block. Inverted hybrid oligonucleotides are described in U.S. Pat. No. 5,652,356, which is hereby incorporated by reference. Generally, such hybrid oligonucleotides comprise regions of deoxyribonucleoside phosphodiesters or phosphorothioates, which flank one or more regions of 2′-O-substituted nucleosides, which may be connected to each other and to the region of deoxyribonucleoside phosphodiesters or phosphorothioates by any type of internucleoside linkage. Thus, in this aspect of the invention, the invention comprises the improvement in an inverted hybrid oligonucleotide of having a POPS block as the region of deoxyribonucleoside phosphodiesters or phosphorothioates.

### BRIEF DESCRIPTION OF THE DRAWINGS

FIG. _1 shows ³¹p NMR and MALDI-TOF MS spectra of oligo 6 (SEQ ID NO: 7). Underlined letters represent deoxynucleosides; plain letters represent 2′-O-methyiribonucleosides; S and O represent phosphorothioate and phosphodiester linkages, respectively.

FIG. 2 shows CGE profiles of comparative stability of oligos 1, 2 and 6 (SEQ ID NOS: 2, 3 and 7) towards SVPD (0.004 units/50 μl) at 37° C. for 24 hr. Intact oligo 1 (SEQ ID NO: 2) was approximately 34%. Peak at 16 min. is of internal standard (PS-oligo 25-mer) added after digestion and before CGE analysis.

FIG. 3 shows RNase H hydrolysis pattern of the 5′-³²P-labeled RNA phosphodiester 30-mer (SEQ ID NO: 1) (5′ ACCGCCGCCAGUGAGGCACGCAGCCUU3′) in the presence of oligos 1 to 6 (SEQ ID NOS: 2 and 7). Lane −T1, control lane without RNase T1 added; lane +T1, RNase T1 digestion reaction; lane —OH, alkaline hydrolysis reaction; lane-DNA, control RNA lane without any oligo added; lanes oligos 1 to 6 (SEQ ID NOS: 2 to 7), in the presence of oligos 1 to 6 (SEQ ID NOS: 2 to 7). respectively and RNA and RNase H. There was no cleavage in presence of oligos 3, 4 and 5 (SEQ ID NOS: 4, 5 and 6) as they are not substrate for RNase H. Lane oligo X is a treatment in the presence of an oligo which is not included in this disclosure. The structure of the oligos is depicted in Table 1.

FIG. 4 shows a comparison of the effects of oligos 1 to 6 (SEQ ID NOS: 2 and 7) on prolongation of aPTT using human blood from healthy volunteer. Each aPTT value is the average of 4 measurements.

FIG. 5 shows CGE profiles of extracted samples of oligo 1(B) (SEQ ID NO: 2) and oligo 6(D) (SEQ ID NO: 7) from mice plasma at 1 hr post-dosing following IV administration.

Panel A and C are control oligo 1 and 6 (SEQ ID NOS: 2 and 7). Peak at 15.5 min. is internal control (PS-oligo 25-mer).

inferred from the observation of specific gene expression inhibition. The gene sequence or RNA transcript sequence to which the modified oligonucleotide sequence is complementary will depend upon the biological effect that is sought to be modified. In some cases, the genomic region, gene, or RNA transcript thereof may be from a virus. Preferred viruses include, without limitation, human immunodeficiency virus (type 1 or 2), influenza virus, herpes simplex virus (type 1 or 2), Epstein-Barr virus, cytomegalovirus, respiratory syncytial virus, influenza virus, hepatitis B virus, hepatitis C virus and papilloma virus. In other cases, the genomic region, gene, or RNA transcript thereof may be from endogenous mammalian (including human) chromosomal DNA. Preferred examples of such genomic regions, genes or RNA transcripts thereof include, without limitation, sequences encoding vascular endothelial growth factor (VEGF), beta amyloid, DNA methyltransferase, protein kinase A, ApoE4 protein, p-glycoprotein, c-MYC protein, BCL-2 protein, protein kinase A and CAPL. In yet other cases, the genomic region, gene, or RNA transcript thereof may be from a eukaryotic or prokaryotic pathogen including, without limitation, Plasmodium falcipa um, Plasmodium malarie, Plasmodium ovale, Schistosoma spp., and Mycobacterium tuberculosis.

The following examples are intended to further illustrate certain preferred embodiments of the invention and are not intended to be limiting in nature. To carry out the studies, we chose a PS-oligo (18-mer, oligo 1, (SEQ ID NO: 2) Table 1) that is complementary to the RIα regulatory subunit of protein kinase A. Oligo 1 (SEQ ID NO: 2) has been studied extensively in both in vitro and in vivo models. In our previous efforts to improve the therapeutic potential of oligo 1 (SEQ. ID NO: 2), we have studied a MBO (oligo 2 (SEQ ID NO: 3)), in which four deoxynucleosides from both 3′- and 5′-ends were substituted with 2′-O-methylribonucleosides. Oligo 2 has the anti-tumor activities similar to those of oligo 1 (SEQ ID NO: 2), but with a significant improvement in pharmacokinetic and toxic profiles observed in mice and rats. Reduction of PS-oligo-related side effects has also been observed. Oligo 2 (SEQ ID NO: 3) is presently being evaluated for its therapeutic potential in human clinical trials.

TABLE 1

Structures of oligos used in this study
and their various parameters

| | | | | |
|---|---|---|---|---|
| | | | Tm | APTT |
| | SEQ | | with | 50% |
| Oligo | ID | | RNA | conc. |
| No. | NOS. | Sequence & Modifications | (° C.) | (μg/ml) |

| | | | | |
|---|---|---|---|---|
| 1 | 2 | 5′ GsCsGsTsGsCsCsTsCsCsTsCsAsCsTsGsGsC 3′ | 62.9 | 37.1 |
| 2 | 3 | 5′ GsCsGsUsGsCsCsTsCsCsTsCsAsCsUsGsGsC 3′ | 72.1 | 46.6 |
| 3 | 4 | 5′ GsCsGsUsGsCsCsUsCsCsUsCsAsCsUsGsGsC 3′ | 84.8 | 81.9 |
| 4 | 5 | 5′ GoCoGoUoGoCoCoUoCoCoUoCoAoCoUoGoGoC 3″ | 87.4 | >200 |
| 5 | 6 | 5′ GsCoGsUoGsCoCsUoCsCoUsCoAsCoUsGoGsC 3′ | 87.2 | >200 |
| 6 | 7 | 5′ GsCoGsUoGsCsCsTsCsCsTsCsAsCoUsGoGsC 3′ | 77.3 | 94.1 |

O—phosphodiester linkage,
s—phosphorothioate linkage,
underlined—deoxynucleoside,
normal—2″-O-methylribonucleoside.

### EXAMPLE 1

### Design of Oligonucleotides

Based on the design of oligo 2 (SEQ ID No: 3), our approach to further minimize the prolongation of aPTT was to reduce the number of phosphorothioate linkages in oligo 2 (SEQ ID NO. 3) without compromising the stability towards nucleases. To carry out the studies, first we designed and prepared some model oligonucleotides (Table 1) to provide insights into the relationship between the nature of the olgonucleotides (nucleoside sugar and phosphate backbone) and its impact on nuclease stability and thermodynamic stability with target RNA, and most importantly, the PS-oligo-related side effects. The oligonucleotides were synthesized using β-cyanoethyl phosphoramidite chemistry on a 15 μmol scale (Expedite 8909, Perceptive Biosystems, MA) or on a 0.5 mmol scale (Pharmacia OligoPilot II Synthesizer). The 2′-O-methyl RNA segments with alternative PS/PO internucleotide linkages in oligos 4, 5 and 6 (SEQ ID NOS: 5, 6 and 7) were synthesized by applying the appropriate oxidation reagents in the corresponding synthesis cycles (Beacauge Reagent for PS linkage, and iodine for PO linkage). The oligos were purified by preparative reverse-phase HPLC. The oligo products were characterized by CGE, ³¹PNMR, and MALDI-TOF MS. These model oligonucleotides included 2′-O-methyloligoribonucleoside phosphorothioate (oligo 3) (SEQ ID NO: 4), 2′-O-methyloligoribonucleoside phosphodiester (oligo 4) (SEQ ID NO: 5) and 2′-O-methyloligoribonucleoside containing alternative phosphorothioate and phosphodiester linkages (oligo 5) (SEQ ID NO: 6).

### EXAMPLE 2

### Stability Of Oligonucleotides

In a study to examine the in vitro stability of the oligos towards snake venom phosphodiesterase (SVPD), the following experiments were performed. For each reaction, oligo (0.5 A₂₆₀, units) was suspended in buffer (50 μl) containing Tris (pH 8.5, 30 mM) and MgCl₂ (15 mM). To each solution, 0.004 units of SVPD from crotallus durissus (Boehringer (Mannheim) was added. The reaction was carried out for 24 hr. at 37° C. The stability of oligos 1 to 5 (SEQ ID Nos: 2 to 7)₋is found to be in the order—oligo 3 (SEQ ID NO: 4)≈oligo 2 (SEQ ID NO: 3)≈oligo 5 (SEQ ID NO: 6)>oligo 1 (SEQ ID NO: 2)₋>>oligo 4 (SEQ ID NO: 5). These results suggest that substitution of one phosphorothioate linkage with a phosphodiester in the 2′-O-methylribonucleoside at alternative sites does not adversely affect the stability of oligo 5 (SEQ ID NO: 6) towards SVPD, compared with that of oligo 3 (SEQ ID NO: 4). In a parallel study, it was found that substitution of the phosphorothioate linkage with a phosphodiester linkage in the PS-oligo (oligo 1 (SEQ ID NO: 2), Table 1) reduced the modified oligos' stability towards SVPD (data not shown).

### EXAMPLE 3

### Stability and Duplex Formation of a POPS Block-Containing Oligonucleotide

Prompted by the above observation, and the data described later, we designed and prepared a new type of MBO—oligo 6 (SEQ ID NO: 5) (Table 1), which contains a PS-oligo segment (nine deoxynucleosides) in the center flanked by five and four 2′-O-methylribonucleosides at both the 3′- and 5′-ends containing alternative phosphorothioate and phosphodiester linkages. The structural nature of oligo 6 (SEQ ID NO: 7) was confirmed by ³¹P NMR and MALDI-TOF MS analysis (FIG. 1).

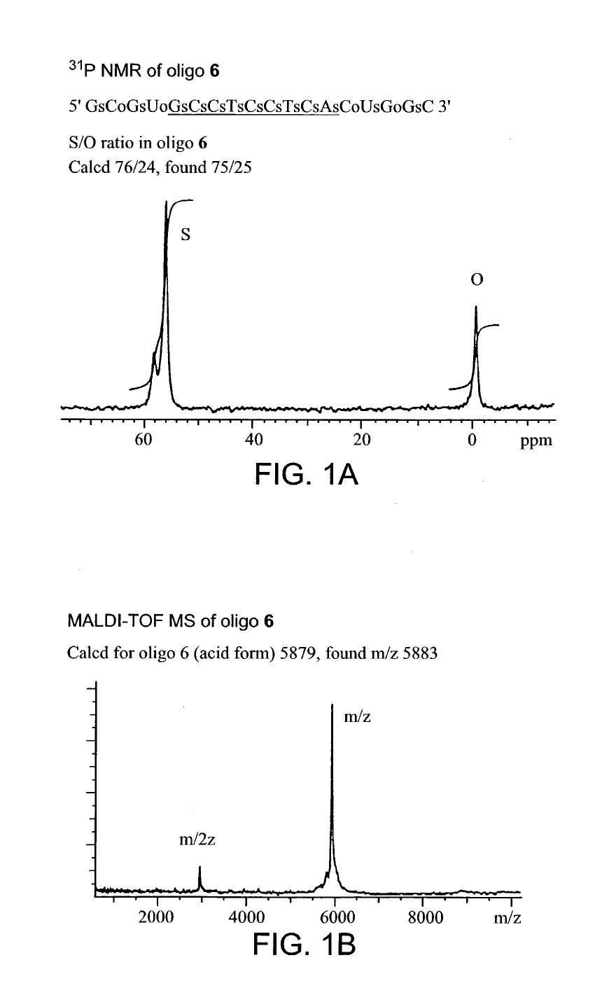

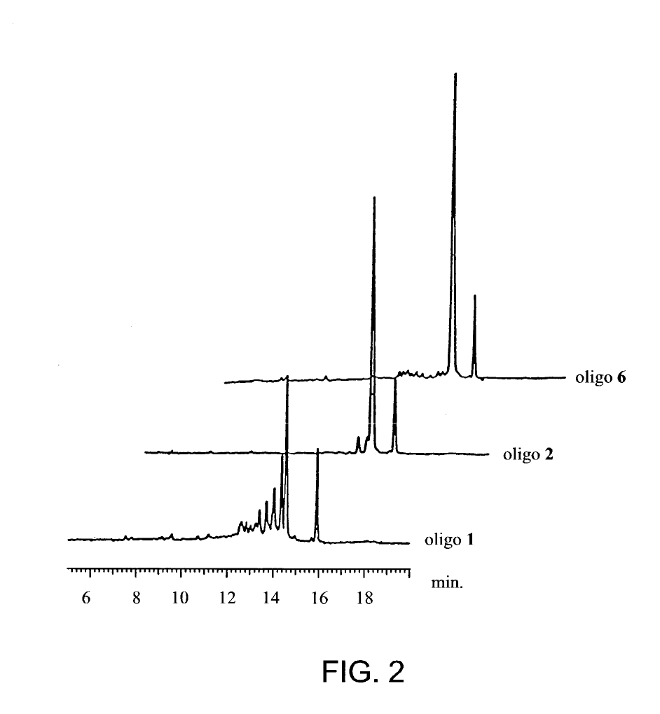

In the study to compare the in vitro stability of the oligos toward SVPD, nuclease resistance was assessed as described in Example 2. Oligo 6 (SEQ ID NO: 7) was found to have stability similar to that of oligo 2 (SEQ ID NO: 3), and have greater stability than oligo 1 (SEQ ID NO: 2). (FIG. 2). This indicated the structural design of oligo 6 (SEQ ID NO: 7) had no adverse effects on the oligo's nuclease stability in vitro.

In the melting temperature (Tm) study to compare the oligos' binding affinity to the complementary RNA phosphodiester, Tm were recorded using a GBC 920 Spectrophotometer (GBC Scientific Equipment, Victoria, Australia). Oligos were mixed with complementary RNA phosphodiester ((30-mer, 5′ ACG GCC GCC AGU GAG GAG GCA CGC AGC CUU 3′) in a buffer containing 10 mM Pipes, 1 mM EDTA, and 100 mM NaCl. The Tm values were obtained from the first derivative plots. Oligo 6 (SEQ ID NO: 7) showed an increase of 14.4° C. and 5.2 ° C. in Tm compared with oligo 1 (SEQ ID NO: 2) and oligo 2 (SEQ ID NO: 3) respectively (Table 1). Compared with oligo 2 (SEQ ID NO: 3), the increase of the binding affinity of oligo 6 (SEQ ID NO: 7), as demonstrated by the increase of Tm, is due to the substitution of four phosphorothioate linkages with phosphodiester linkages and also an additional 2′-O-methylribonudeoside.

### EXAMPLE 4

### Rnase H Activation by a POPS Block-Containing Oligonucleotide

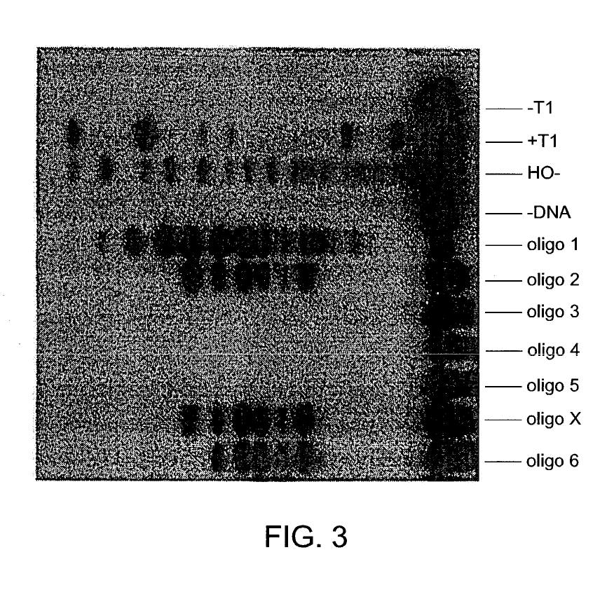

RNase H digestion studies were carried out as follows. For each reaction, the 5′-³²p-labeled RNA phosphodiester (30-mer, 0.5 pmol), oligo (5 pmol), and glycogen (50 μmol) were mixed in 12 μl of buffer containing 50 mM MgCl₂, 100 mM KCl, 1 mM DTT, 200 mM Tris (pH 7.5), and 5% glycerol. Aftere annealing, 0.078 unit of RNase H (Pharmacia) was added to each solution. The mixture were then incubated at 37° C. for 10 min. The reactions were then quenched by adding 20 μl of gel loading dye to each reaction mixture. The resultant samples were analyzed by 20% PAGE and subjected to autoradiography. Oligos 2 and 6 (SEQ ID NOS: 2 and 7) showed to have similar cleavage patterns, which differed from that of oligo 1 (SEQ ID NO: 2) due to the flanking 2′-O-methylribonucleosides in oligos 2 and 6 (SEQ ID Nos: 3 and 7) (^{ref}. 1) (FIG. 3). This study indicated that the MBO design of oligo 6 (SEQ ID NO: 7) had no adverse impact on the oligo's ability to cleave the complementary RNA in presence of RNase H.

### EXAMPLE 5

### PS-Mediated Side Effects of a POPS Block-Containing Oligonucleotide

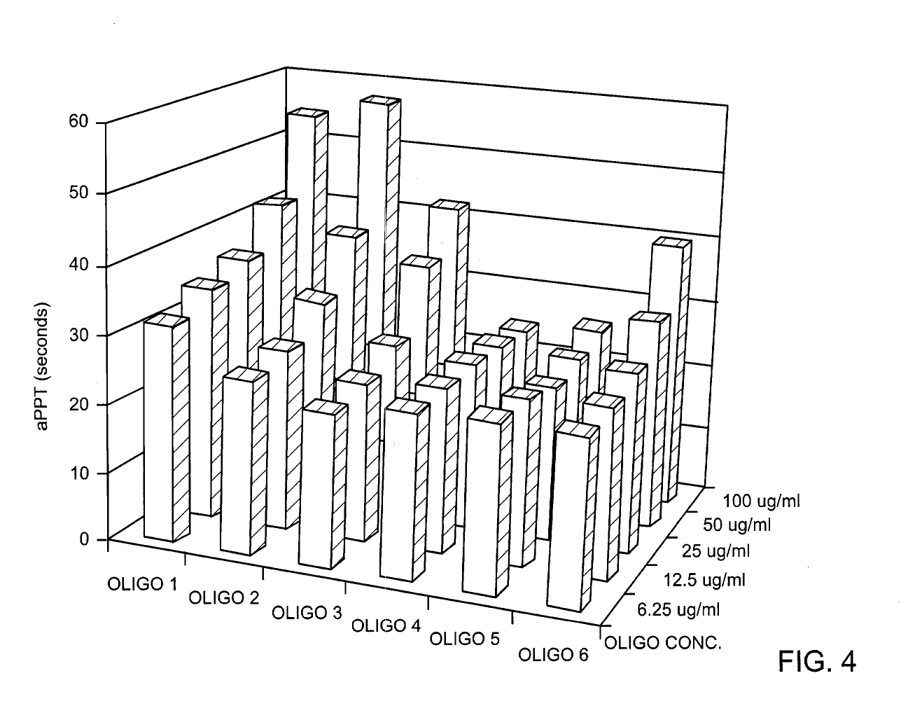

Compared with oligo 2 (SEQ ID NO: 3), this newly-designed MBO (oligo 6 (SEQ ID NO: 7)) has less phosphorothioate content, and thus may have less PS-oligo-related side effects. Next, the effects of oligos 1 to 6 (SEQ ID NOS: 2 to 7)₃₁ on prolongation of aPTT were compared. The study was to see if oligo 6 (SEQ ID NO: 7) with a reduced number of phosphorothioate linkages was indeed able to reduce the PS-oligo-related side effects such as prolongation of aPTT. Plasma was obtained from citrated human blood. Serial dilution of the oligos in 0.9% NaCl UPS (saline) were made to provide final concs. of 6.25, 12.5, 25, 50 and 100 μg/ml of oligo in plasma. After addition of the oligo samples, the plasma was incubated at 37° C. for 15 min., with gentle agitation. Plasma exposed to vehicle in the same ratio (v/v) as the oligos, and untreated plasma served as negative controls. The assay was conducted in duplicate, providing at least 2 replication for each tube. The aPTT test was performed by TOXICON (BEDFORD, MD.). The results are depicted in FIG. 4. All oligos showed concentration-dependent prolongation of aPTT, but with significant differences among the oligos. The clear differences between oligo 1 (SEQ ID NO: 2) (PS-oligo) and oligo 3 (SEQ ID NO: 4) (2′-O-methyloligoribonucleoside phosphorothioate) confirmed our previous observation that phosphorothioate linkage of the oligodeoxynucleoside (PS-oligo) is more effective in prolonging the aPTT than the phosphorothioate linkage of the oligoribonucleoside analogs, including 2′-O-methylribonucleoside. As expected, oligos 4 and 5 (SEQ ID NOS: 5 and 6) showed the least prolongation of aPTT, due to the dominant content of the 2′-O-methylribonucleoside and the least content of phosphorothioate linkages (Table 1). The concentration required for oligos 4 and 5 (SEQ ID NOS: 5 and 6) to prolong 50% aPTT was more than 200 μg/ml (>35 μM). In general, the prolongation of aPTT in presence of oligos 1 to 6 (SEQ ID NOS: 2 to 7) was in the order—oligo 1 (SEQ ID NO: 2)>oligo 2 (SEQ ID NO: 3)>oligo 3 (SEQ ID NO: 4)>oligo 6 (SEQ ID NO: 7)>oligo 4 (SEQ ID NO: 5)₃₁ >oligo 5 (SEQ ID NO: 6). To our satisfaction, oligo 6 (SEQ ID NO: 7)—the newly-designated MBO in which flanking sequences contain 2′-O-methylribonucleosides with alternative phosphorothioate and phosphodiester linkages—showed a significant reduction in its ability to prolong aPTT, compared with oligos 1 and 2 (SEQ ID NOS.: 2 and 3). The concentration required to prolong aPTT by 50% for oligos 1, 2, and 6 (SEQ ID NOS.: 2, 3 and 7) was 37.1, 46.6 and 94.1 μg/ml, respectively (Table 1).

### EXAMPLE 6

### In Vivo Stability of a POPS Block-Containing Oligonucleotide

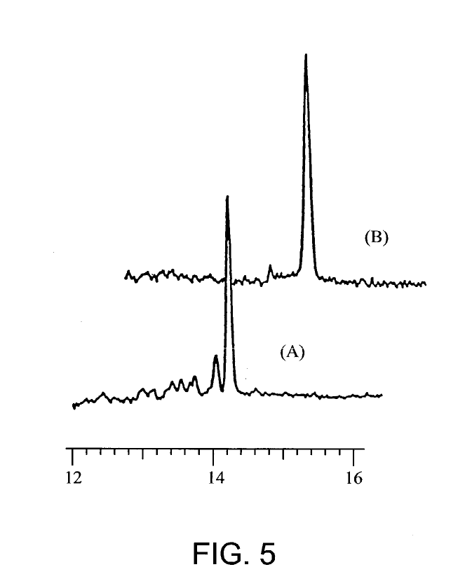

Prompted by the above in vitro results, we extended our study to compare the in vivo stability of oligo 6 (SEQ ID NO: 7) with that of oligo 1 (SEQ ID NO: 2). Oligo 1 and 6 (SEQ ID NOS: 2 and 7) (1 mg) were administered intravenously in mice (female, CD-1, 20-22 g) through the tail vein. Following intravenous administration on these two oligos in mice, blood samples were drawn from mice at the post-dosing time points of 30 min., 1, 12 and 24 hours. The oligo components were then carefully extracted from the plasma. Part of the oligo samples was analyzed by 20% polyacrylamide gel electrophoresis (PAGE) after the 5′-end labeling with ³²P, and part of the oligo samples was subjected to direct CGE analysis (with a UV detector). The PAGE autoradiograph showed presence of bands representing intact length of oligo 6 (SEQ ID NO: 7) at much longer time points compared with oligo 1 (SEQ ID NO: 2) (data not shown). The increased in vivo stability of oligo 6 (SEQ ID NO: 7), compared with oligo 1 (SEQ ID NO: 2), was also confirmed by the CGE analysis. The CGE profile of oligo 1 (SEQ ID NO: 7) showed approximately 55% intact oligo and 45% in degraded form, where as majority of oligo 6 (SEQ ID NO: 7) was in intact form (FIG. 5). In conclusion, our studies demonstrate that it is possible to optimize the properties of antisense oligos by subtle structural changes in the nucleoside sugar residue and intemucleotide, as exemplified by the design of oligo 6 (SEQ ID NO: 7). Our preliminary pharmacokinetic study also showed that the tissue disposition profile of oligo 6 (SEQ ID NO: 7) is similar to that of oligo 2 (SEQ ID NO: 3), which suggests that reduction of the phosphorothioate linkages in oligo 6 (SEQ ID NO: 7)₋does not result in significant changes in tissue deposition (data not shown). Other studies are ongoing to fully exploit the therapeutic potential of oligo 6 (SEQ ID NO: 7). Similar design of antisense oligos is applying to other disease models.

Recommended Literature

1. Agrawal, S. Trends Biotechnol., 1996, 14, 376.

2. Altmann, K; Dean, N.; Fabbro, D.; Freier, S.; Geiger, T.; Haner, R.; Husken, D.; Martin, P.; Monia, B.; Muller, M.; Natt, F.; Nicklin, P.; Phillips, J.; Pieles, U.; Sasmor, H.; Moser, H. Chimia, 1996, 50, 168.

3. (a) Agrawal, S.; Mayrand, S.; Zamecnik, P.; Pederson, T. Proc. Natl. Acad Sci. USA, 1990, 87, 1401. (b) Devlin, T.; Iyer, R; Johnson, S.; Agrawal, S. Bioorg. Med. Chem. Lett., 1996, 6, 2663. (c) Giles, R.; Spiller, D.; Tidd, D., Antisense Res. Dev., 1995, 5, 23. (d) Iyer, R.; Yu, D.; Jiang, Z.; Agrawal, S. Tetrahedron, 1996, 52, 14419.

4. (a) Metelev, V.; Lisziewicz, J.; Agrawal, S. Bioorg. Med. Chem. Lett., 1994, 4, 2929. (b) Metelev, V.; Agrawal, S. Proceeding of International Conferences on Nucleic Acid Medical Applications, Cancun, January 1993, Abstract 1-1. (c) Monia B.; Lesnik, E.; Gonzalez, C.; Lima, W.; McGee, D.; Guinosso, C.; Kawasaki, A.; Cook. P. J. Biol. Chem., 1993, 268, 14514. (d) Yu, D.; Iyer, R.; Shaw, D.; Lisziewicz, J.; Li, Y.; Jiang, Z.; Roskey, A.; Agrawal, S. Bioorg. Med. Chem., 1996,4, 1685.

5. Zhao, Q.; Temsamani, J.; ladarola, P.; Jiang, Z.; Agrawal, S. Biocheml Pharmacol., 1996, 51, 173.

6. Shaw, D.; Rustagi, P.; Kandimalla, E.; Manning, A.; Jiang, Z.; Agrawal, S. Bioch. Pharmacol., 1997, 53, 1123.

7. Agrawal, S.; Jiang, Z. Zhao, Q.; Shaw, D.; Cai, Q.; Roskey, A.; Channavajjala, L.; Saxinger, C.; Zhang, R. Proc. Natl. Acad Sci. USA, 1997, 94, 2620.

8. Zhang, R.; Lu, Z.; Liu, T.; Zhao, H.; Zhang, X.; Diasio, S.; Habus, I.; Jiang, Z.; Iyer, R.; Yu, D.; Agrawal, S. Biochem. Pharnacol., 1995, 50, 545.

9. Agrawal, S.; Zhang, X.; Zhao, H.; Lu, Z.; Yan, J.; Cai, H.; Diasio, R.; Habus, I.; Jiang, Z.; Iyer, R.; Yu, D.; Zhang, R. Biochem. PharmacoL, 1995, 50, 571.

10. Nesterova, M.; Cho-Chung, Y. Nat. Med. 1995, 1, 528.

11. Agrawal, S.; Zhao, Q. Antisense Res. Dev., in press.

12. Grindel, J.; Musick, T.; Jiang, Z.; Roskey, Al; Agrawal, S. Antisense Res. Dev., 1998, 8, 43.

What is claimed is:

1. An improved antisense oligonucleotide, the improvement comprising the presence of one or more POPS block.

2. The improved antisense oligonucleotide according to claim 1, wherein the oligonucleotide is a hybrid oligonucleotide.

3. The improved antisense oligonucleotide according to claim 1, wherein the oligonucleotide is an inverted hybrid oligonucleotide.


SEQUENCE LISTING (8 sequences)

SEQ ID NO: 1 - RNA, 27 nt, Artificial Sequence
  RNA phosphodiester
accgccgccagugaggcacgcagccuu

SEQ ID NO: 2 - DNA, 18 bp, Artificial Sequence
  Oligo No. 1
gcgtgcctcctcactggc

SEQ ID NO: 3 - DNA, 18 bp, Artificial Sequence
  Oligo No. 2
gcgugcctcctcacuggc

SEQ ID NO: 4 - DNA, 18 bp, Artificial Sequence
  Oligo No. 3
gcgugccuccucacuggc

SEQ ID NO: 5 - DNA, 18 bp, Artificial Sequence
  Oligo No. 4
gcgugccuccucacuggc

SEQ ID NO: 6 - DNA, 18 bp, Artificial Sequence
  Oligo No. 5
gcgugccuccucacuggc

SEQ ID NO: 7 - DNA, 18 bp, Artificial Sequence
  Oligo No. 6
gcgugcctcctcacuggc

SEQ ID NO: 8 - RNA, 30 nt, Artificial Sequence
  RNA phosphodiester
acggccgccagugaggaggcacgcagccuu